(19) United States
(12) Patent Application Publication
(10) Pub. No.: US 2022/0039693 A1
(43) Pub. Date: Feb. 10, 2022

(54) BODY MASS INDEX INTERVAL ESTIMATION DEVICE AND OPERATION METHOD THEREOF

(71) Applicant: Merry Electronics(Shenzhen) Co., Ltd., ShenZhen (CN)

(72) Inventors: Yen-Sheng Chen, Taichung (TW); Ping Hung Chu, Taichung (TW)

(73) Assignee: Merry Electronics(Shenzhen) Co., Ltd., ShenZhen (CN)

(21) Appl. No.: 17/019,321

(22) Filed: Sep. 13, 2020

(30) Foreign Application Priority Data
Aug. 5, 2020 (TW) 109126584

(51) Int. Cl. A61B 5/11; A61B 5/00

(52) U.S. Cl. CPC A61B 5/112; A61B 5/7264; A61B 5/6813; A61B 2562/0219; A61B 5/7405; A61B 5/4872; A61B 5/742

(57) ABSTRACT

A body mass index interval estimation device including an inertial sensor and an arithmetic circuit is provided. The inertial sensor is suitable for being worn on a body part to detect and obtain first gait information of a user in a traveling state. The first gait information includes first three-axis acceleration information and first three-axis angular velocity information. The arithmetic circuit causes processed first gait information after a pre-processing to pass an identification model to generate first body mass index interval information. The identification model is created by performing a training on a sample database. The sample database includes a plurality of second gait information and a plurality of label information corresponding thereto in a one-to-one manner. Each second gait information further includes second three-axis acceleration information and second three-axis angular velocity information.

### CROSS-REFERENCE TO RELATED APPLICATION

This application claims the priority benefit of Taiwan application no. 109126584, filed on Aug. 5, 2020. The entirety of the above-mentioned patent application is hereby incorporated by reference herein and made a part of this specification.

### TECHNICAL FIELD

The invention relates to a body mass index interval estimation device, and more particularly, relates to a device for estimating a body mass index interval based on user's gait information.

### BACKGROUND

One of indicators to measure the degree of obesity is the BMI (Body Mass Index). Weight (in kilograms) divided by the square of height (in meters) is the BMI. In general, the BMI of healthy adults should be greater than or equal to 18.5 and less than 24. The BMI less than 18.5 indicates underweight. The BMI that falls in an interval greater than or equal to 24 and less than 27 indicates overweight. The BMI that falls in an interval greater than or equal to 27 and less than 30 indicates mild obesity; the BMI that falls in an interval greater than or equal to 30 and less than 35 indicates moderate obesity; the BMI that falls in an interval greater than or equal to 30 and less than 35 indicates severe obesity.

Underweight, overweight, or various degrees of obesity deviate from the normal range of health. Studies have shown that overweight or obesity is the main risk factor for chronic diseases such as diabetes, cardiovascular disease, and malignant tumors. The health problems regarding underweight include malnutrition, osteoporosis, and sudden death. However, the weight used to calculate the body mass index will vary with diet, lifestyle habits, and measurement time. Therefore, it is necessary to measure the weight constantly to know which interval the body mass index falls within. However, due to many factors such as time, site and persistence, most people may not be able to measure their weight regularly and calculate the corresponding BMI.

Therefore, it is necessary to provide a solution that can automatically estimate current body mass index interval information in real time.

### SUMMARY

The invention provides a body mass index interval estimation device and an operation method thereof, which are capable of providing the current body mass index interval information in real time.

The body mass index interval estimation device includes an inertial sensor and an arithmetic circuit. The inertial sensor is suitable for being worn on a body part to detect and obtain first gait information of a user in a traveling state. The first gait information includes first three-axis acceleration information and first three-axis angular velocity information. The arithmetic circuit is configured to cause processed first gait information after a pre-processing to pass an identification model to generate first body mass index interval information. The identification model is created by performing a training on a sample database. The sample database includes a plurality of second gait information and a plurality of label information corresponding thereto in a one-to-one manner. Each second gait information further includes second three-axis acceleration information and second three-axis angular velocity information.

The operation method of the body mass index interval estimation device includes: detecting and obtaining first gait information of a user in a traveling state by the inertial sensor worn on a body part, wherein the first gait information includes first three-axis acceleration information and first three-axis angular velocity information; and causing processed first gait information after a pre-processing to pass an identification model by the arithmetic circuit to generate first body mass index interval information. The inertial sensor and the arithmetic circuit are included in the body mass index interval estimation device. The identification model is created by performing a training on a sample database. The sample database includes a plurality of second gait information and a plurality of label information corresponding thereto in a one-to-one manner. Each second gait information further includes second three-axis acceleration information and second three-axis angular velocity information.

Based on the above, the invention can be used to collect the acceleration information and the angular velocity information and estimate the current body mass index interval information of the user through the identification model. In this way, the current body mass index interval information can be obtained in real time simply by walking without being restricted by the measurement time and site.

### BRIEF DESCRIPTION OF THE DRAWINGS

FIG. 1 is a block diagram of a body mass index interval estimation device according to an embodiment of the invention.

FIG. 2 is a schematic diagram of an operation method of a body mass index interval estimation device according to an embodiment of the invention.

FIG. 3 is a schematic diagram of flows in a pre-processing according to an embodiment of the invention.

FIG. 4 is a flowchart illustrating steps of an operation method of a body mass index interval estimation device according to an embodiment of the invention.

FIG. 5 is a schematic diagram of a first modification of the operation method of the body mass index interval estimation device.

FIG. 6 is a schematic diagram of a second modification of the operation method of the body mass index interval estimation device.

### DETAILED DESCRIPTION

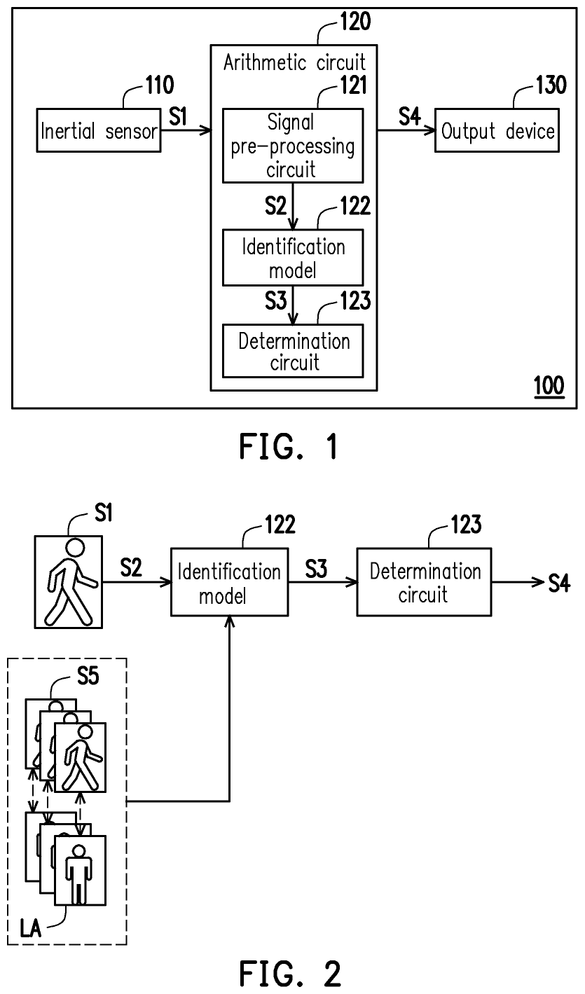

FIG. 1 is a block diagram of a body mass index interval estimation device according to an embodiment of the invention. Referring to FIG. 1, a body mass index interval estimation device 100 includes an inertial sensor 110, an arithmetic circuit 120 and an output device 130. The inertial sensor 110 is suitable for being worn on a body part of a user. In this embodiment, the inertial sensor 110 can be used as a part of an earphone and worn on the ear. In other embodiments, the inertial sensor 110 may also be worn on the waist, hands or legs.

The inertial sensor 110 can detect and obtain first gait information s1 of the user in a traveling state. Gait is a compound result of the complex interaction of the body's complex muscles, bones, nerves and even joints. According to Newtonian mechanics, the actions of muscles, bones and other elements are all related to force, moment, acceleration, angular velocity, and the mass and geometry of the corresponding motion element. Therefore, gait information essentially contains relevant characteristic information such as mass, acceleration, angular velocity and force. The invention proposes to collect data through the inertial sensor in the earphone (or other wearable products) worn on the body, and to determine a body mass index interval of the user through an identification model.

In this embodiment, the inertial sensor 110 may include a 3-axis accelerometer and a 3-axis angular velocity meter, which are configured to obtain the first gait information s1 including three-axis acceleration information and three-axis angular velocity information. The 3-axis accelerometer usually uses a position measurement interface circuit to measure the displacement of an object, and then uses an analog-to-digital converter (ADC) to convert a measured value into a digital electronic signal for digital processing. The 3-axis angular velocity meter (e.g., a 3-axis gyroscope) can measure the resonance and displacement of an object due to Coriolis acceleration.

In other embodiments, in addition to the 3-axis accelerometer and the 3-axis gyroscope, the inertial sensor 110 may also include a 3-axis magnetometer. The structures and functions of the accelerometer and the angular velocity meter are well-known to those with ordinary knowledge in the field to which the invention belongs, and thus will not be repeated herein.

The arithmetic circuit 120 is coupled to the inertial sensor 110 to receive the first gait information s1 including the three-axis acceleration information and the three-axis angular velocity information. The arithmetic circuit 120 may include a signal pre-processing circuit 121, an identification model 122 and a determination circuit 123. The signal pre-processing circuit 121 is configured to perform a pre-processing on signals detected by the inertial sensor 110 to obtain processed first gait information s2. The arithmetic circuit 120 is configured to generate an identification result s3 according to the processed first gait information s2 through the identification model 122. According to the identification result s3, the determination circuit 123 then generates first body mass index interval information for the output device 130 (a display, a speaker or other types of output devices) to output.

FIG. 2 is a schematic diagram of an operation method of a body mass index interval estimation device according to an embodiment of the invention. Referring to FIG. 1 and FIG. 2 together, the first gait information s1 of the user in the traveling state is detected and obtained by the inertial sensor 110 worn on the body part. The first gait information s1 includes the three-axis acceleration information and the three-axis angular velocity information. Then, the arithmetic circuit 120 generates the identification result s3 according to the processed first state information s2 after the pre-processing through the identification model 122. Lastly, first body mass index interval information s4 is generated according to the identification result s3 by the determination circuit 123.

It should be noted that, the identification model 122 is created by performing a training on a sample database. The sample database includes a plurality of second gait information s5 and a plurality of label information LA. There is a one-to-one relationship between the plurality of second gait information s5 and the plurality of label information LA. Each of the second gait information includes three-axis acceleration information and three-axis angular velocity information. Each of the label information LA is corresponding second body mass index interval information. It should be noted that both the first gait information and the second gait information are obtained by the inertial sensor worn on the same body part of the user.

In terms of hardware, the signal pre-processing circuit 121, the identification model 122, and the determination circuit 123 may be logic circuits implemented on an integrated circuit. The related functions of the pre-processing circuit 121, the identification model 122 and the determination circuit 123 may be implemented as hardware using hardware description languages (e.g., Verilog HDL or VHDL) or other suitable programming languages. For instance, the related functions of the pre-processing circuit 121, the identification model 122 and the determination circuit 123 may be implemented as various logic blocks, modules and circuits in one or more controllers, microcontrollers, microprocessors, application-specific integrated circuits (ASIC), digital signal processors (DSP), field programmable gate arrays (FPGA) and/or other processing units.

A training process of the sample database will be described as follows. In this embodiment, a plurality of nodes (neurons) in an artificial neural network can be divided into five layers. The nodes between the input layer and the hidden layer are fully connected, and the nodes between the hidden layers are also fully connected. Further, softmax can be used as the activation function of the output layer. The second to fourth layers may include 200, 100 and 80 nodes, respectively. An input quantity of the artificial neural network can be 120 input feature values. The feature values are obtained by a feature calculation on raw data, and an output quantity can be 23 BMI intervals ranged from BMI 14 to BMI 37. The first interval is greater than BMI 14 and less than or equal to BMI 15, the second interval is greater than BMI 15 and less than or equal to BMI 16, and so on and so forth. Correspondingly, for the trained identification model 122, an input quantity is 120 input feature values and an output quantity is 23 BMI intervals. The training process of the artificial neural network of this embodiment adopts Backpropagation (BP) in which the number of layers and the number of nodes used are variable, and the protection range should not be limited thereto. In the training process, each of the second gait information s5 after the pre-processing is sequentially input to the neural network and an output is obtained. A weight between each node is updated according to a difference between the output and the label information LA. The above process is repeated until all data in the sample database are used up. Finally, the trained neural network architecture and the corresponding weight values are implemented on the hardware circuit to obtain the identification model 122. In this embodiment, different outputs of the identification model 122 may correspond to different body mass index intervals.

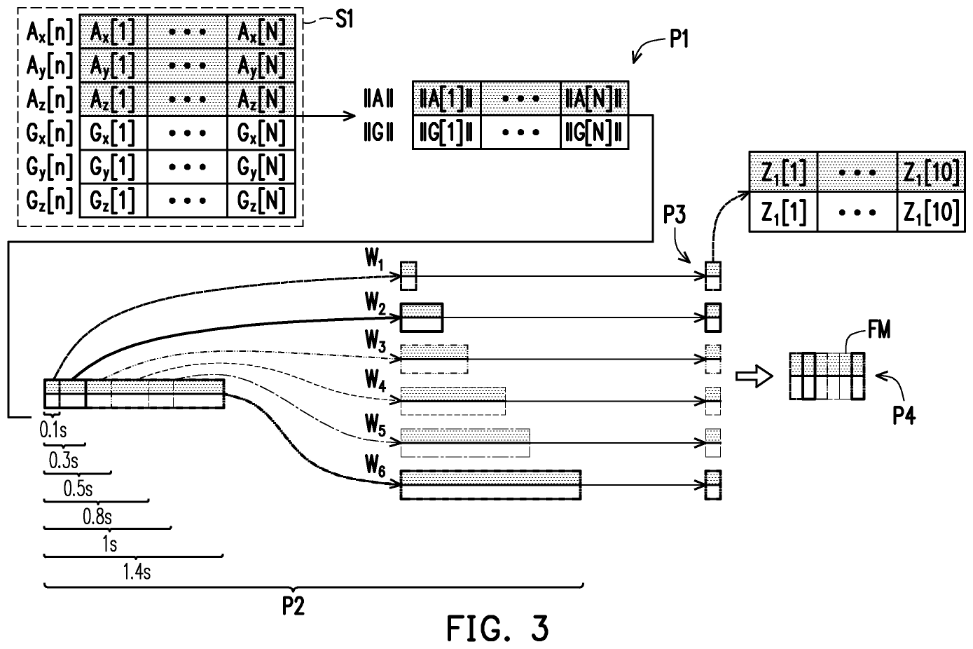

FIG. 3 is a schematic diagram of flows in a pre-processing according to an embodiment of the invention. The raw data provided to the artificial neural network and the identification model 122 need to be pre-processed for a feature extraction. Referring to FIG. 3, the pre-processing can be divided into a first phase P1 to a fourth phase P4. Referring to FIG. 1 and FIG. 3 together, first, the inertial sensor 110 detects and obtains the first gait information s1. The first gait information s1 includes the three-axis acceleration information and the three-axis angular velocity information. The three-axis acceleration information includes acceleration information Aₓ [n], A_{y} [n] and A_{z} [n]. The three-axis angular velocity information includes angular velocity information Gₓ [n], G_{y} [n] and G_{z} [n]. Here, n is a positive integer greater than or equal to 1 and less than or equal to N. N is a positive integer representing a total of data points. A plurality of data included by the acceleration information Aₓ [n] may be denoted as Aₓ [1], . . . , and Aₓ [N]. A plurality of data included by A_{y} [n], A_{z} [n], Gₓ[n], G_{y} [n] and G_{z} [n] can be deduced by analogy.

In the first phase P1, the signal pre-processing circuit 121 is configured to perform the pre-processing on the information Aₓ[n], A_{y} [n], A_{z} [n], Gₓ [n], G_{y} [n] and G_{z} [n] detected by the inertial sensor 110. Specifically, according to Formula (1) and Formula (2), the signal pre-processing circuit 121 can calculate 2-norm of the acceleration information Aₓ [n], A_{y} [n] and A_{z} [n] and the angular velocity information Gₓ[n], G_{y} [n] and G_{z} [n], so as to obtain ∥A∥ and ∥G∥. In this way, ∥A[1]∥, . . . , and ∥A[N]∥ and ∥G[1]∥, . . . , and ∥G[N]∥ may be obtained.

∥A(n)∥≡\sqrt{Aₓ²[n]+A_{y}²[n]+A_{z}²[n]},n=1, . . . ,N Formula (1)

∥G(n)∥≡\sqrt{Gₓ²[n]+G_{y}²[n]+G_{z}²[n]},n=1, . . . ,N Formula (2)

In the second phase P2, the signal pre-processing circuit 121 performs the data extraction on ∥A∥ and ∥G∥ by using six finite impulse response (FIR) filters W₁ to W₆ having windows with different time widths, so as to obtain six extracted data sets. Here, an amount of the extracted data is proportional to the time width of the window. The widths of the six windows are 0.1 second, 0.3 second, 0.5 second, 0.8 second, 1.0 second and 1.4 second, respectively. The FIR filter can be expressed as formula (3), in which x is an input (substituting ∥A∥ and ∥G∥) and yᵢ[n] is an output. In the third phase P3, the signal pre-processing circuit 121 performs a downsampling on filtered data (referring to Formula (4), where yᵢ is an input and zi[m] is an output). The downsampling aims to arrange the filtered data into six matrices of the same size (2×10). The elements in the first row (denoted by Z₁[1], . . . , and Z₁[10]) and the elements in the second row (denoted by Z₂[1], . . . , and Z₂[10]) of each matrix correspond to acceleration features and angular velocity features respectively. 10 is a vector length after the downsampling, which is a preset value adopted by the invention. In Formula (3) and Formula (4), F is the number of filters, and Pᵢ is window points. Sᵢ=P_{MAX}−Pᵢ, which represents the number of points to be translated for different windows. bᵢⱼ is an impulse response of the filter, usually called a coefficient of the filter. Mᵢ=Pᵢ/H, in which H is the vector length after the downsampling. In this embodiment, the FIR filter may be a moving average filter.

yᵢ[n]=Σⱼ₌₀^{M}^{ᵢ}⁻¹bᵢⱼx[n−j−Sᵢ]·i=1, . . . ,F·n=1, . . . ,Pᵢ Formula (3)

zᵢ[m]=Σₖ₌₀^{P}^{ᵢ}⁻¹yᵢ[mMᵢ−k]·h[k]·i=1, . . . ,F·m=1, . . . ,H Formula (4)

In the fourth phase P4, the signal pre-processing circuit 121 combines the six matrices of the same size (2×10) to generate a feature matrix FM (2×60). The 120 elements of the feature matrix FM are used as an input of the artificial neural network and an input of the identification model 122.

In this embodiment, the number of filters (i.e., F) can be 6, and the vector length (i.e., H) after the downsampling can be set to 10. Therefore, Mᵢ=Pᵢ/10, in which Pᵢ is the window points. The coefficient of the filter bᵢⱼ=1/Mᵢ where j=0, . . . , Mᵢ−1. The coefficients of the corresponding filters are shown in Table (1). Because multiple filters with different time widths are used for the data extraction, gait feature information corresponding to different time widths may be captured from a set of gait information.

TABLE 1

| | | | Points | | |
|---|---|---|---|---|---|
| | | | to be | | |
| | | | translated | | |
| Window | Window | Window | (Sᵢ = | Mᵢ | |
| ordinal | interval | points | P_{MAX} − | (=Pᵢ/ | bᵢⱼ |
| (i) | (sec) | (Pᵢ) | Pᵢ) | 10) | (=1/Mi) |
| 1 | 0.1 | 10 | 130 | 1 | b₁₁ = 1 |
| 2 | 0.3 | 30 | 110 | 3 | b₂ⱼ = ⅓ |
| | | | | | (j = 0, . . . , 2) |
| 3 | 0.5 | 50 | 90 | 5 | b₃ⱼ = ⅕ |
| | | | | | (j = 0, . . . , 4) |
| 4 | 0.8 | 80 | 60 | 8 | b₄ⱼ = ⅛ |
| | | | | | (j = 0, . . . , 7) |
| 5 | 1.0 | 100 | 40 | 10 | b₅ⱼ = 1/10 |
| | | | | | (j = 0, . . . , 9) |
| 6 | 1.4 | 140 | 0 | 14 | b₆ⱼ = 1/14 |
| | | | | | (j = 0, . . . , 13) |

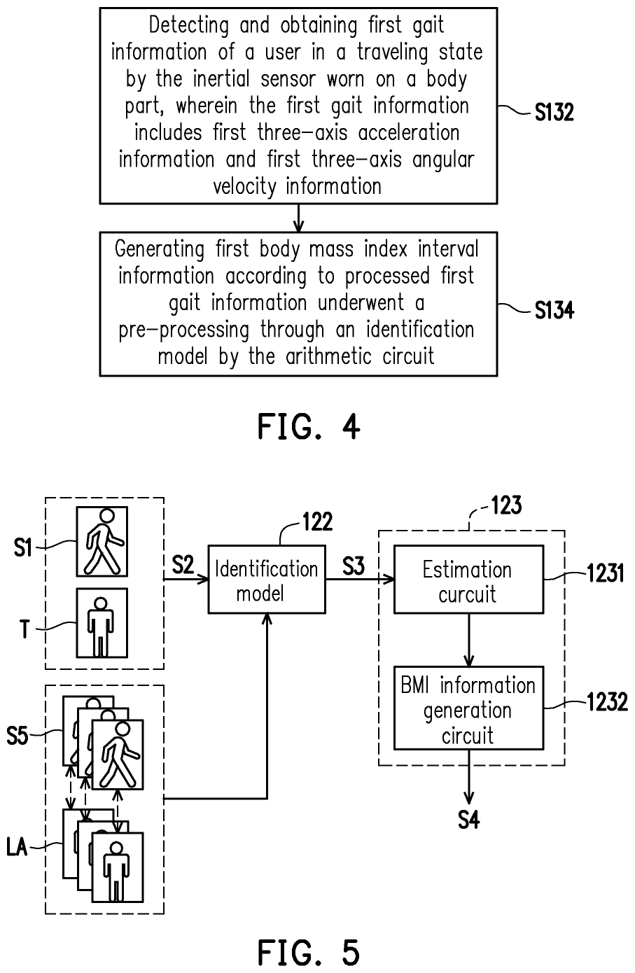

FIG. 4 is a flowchart illustrating steps of an operation method of a body mass index interval estimation device according to an embodiment of the invention. Here, the body mass index interval estimation device includes an inertial sensor and an arithmetic circuit. Referring to FIG. 4, in step S410, first gait information of a user in a traveling state is detected and obtained by the inertial sensor worn on a body part. The first gait information includes first three-axis acceleration information and first three-axis angular velocity information. In step S420, first body mass index interval information is generated according to processed first gait information after a pre-processing through an identification model by the arithmetic circuit. Here, the identification model is created by performing a training on a sample database. The sample database includes a plurality of second gait information and a plurality of label information corresponding thereto in a one-to-one manner. Each of the second gait information further includes second three-axis acceleration information and second three-axis angular velocity information.

Although the invention is implemented with the artificial neural network and the moving average filter in the foregoing embodiment, this should not be a limitation when interpreting the scope of the invention. In other embodiments, those with ordinary knowledge in the field of the invention can use other machine learning algorithms, such as a decision tree, a support vector machine (SVM), a multivariable linear regression, a random decision forests, a convolutional neural network (CNN) and a recurrent neural network (RNN). Moreover, those with ordinary knowledge in the field to which the invention pertains can also use other feature extraction methods. For example, the feature extraction may be performed based on methods such as statistics, nonlinear features, and frequency features. Among them, the statistics are, for example, mean, median, kurtosis, and standard deviation (STD). The nonlinear features are, for example, Lyapunov exponent, fractal dimension, and entropy.

It should be noted that in the foregoing embodiment, each label information of the sample database is body mass index interval information. However, the invention is not limited in this regard. In other modifications, each label information may be weight interval information and height interval information.

FIG. 5 is a schematic diagram of a first modification of the operation method of the body mass index interval estimation device. Referring to FIG. 1 and FIG. 5 together, first, height information T of the user is obtained. The height information T may be provided by the user and pre-stored in a memory circuit of the body mass index interval estimation device 100. Then, first gait information s1 of the user in the traveling state is detected and obtained by the inertial sensor 110 worn on the body part. The first gait information s1 includes the three-axis acceleration information and the three-axis angular velocity information. Then, the arithmetic circuit 120 generates the identification result s3 according to the processed first state information s2 after the pre-processing through the identification model 122. In the first modification, the identification model 122 is also created by performing the training on the sample database. However, unlike the identification model 122 of the embodiment shown in FIG. 2, each label information LA in the sample database of the first modification is not the second body mass index interval information, but the weight interval information and the height interval information.

Lastly, first body mass index interval information s4 is generated according to the identification result s3 by the determination circuit 123. The determination circuit 123 may include an estimation circuit 1231 and a BMI information generation circuit 1232. The BMI information generation circuit 1232 can store a BMI calculation formula. The estimation circuit 1231 can obtain the weight interval information according to the identification result s3. The BMI information generation circuit 1232 generates BMI interval information (i.e., the first body mass index interval information s4) by using the BMI calculation formula according to the weight interval information and the height information T.

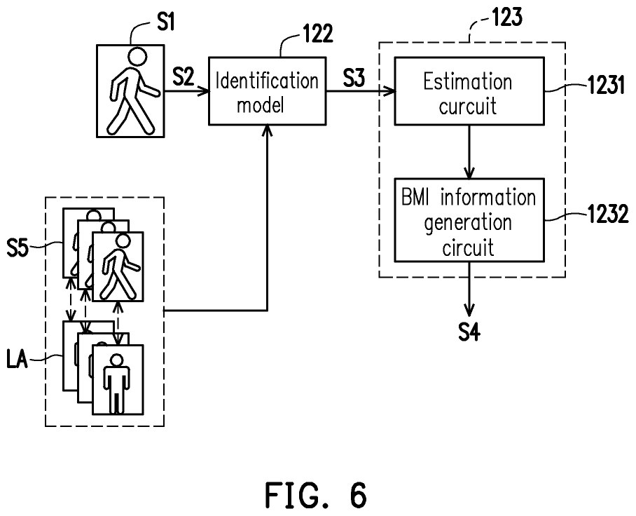

FIG. 6 is a schematic diagram of a second modification of the operation method of the body mass index interval estimation device. Referring to FIG. 1 and FIG. 6 together, the first gait information s1 of the user in the traveling state is detected and obtained by the inertial sensor 110 worn on the body part. The first gait information s1 includes the three-axis acceleration information and the three-axis angular velocity information. Then, the arithmetic circuit 120 generates the identification result s3 according to the processed first state information s2 after the pre-processing through the identification model 122. In the second modification, the identification model 122 is also created by performing the training on the sample database. However, unlike the identification model 122 of the embodiment shown in FIG. 2, each label information LA in the sample database of the second modification is not the second body mass index interval information, but the weight interval information and the height interval information.

Lastly, first body mass index interval information s4 is generated according to the identification result s3 by the determination circuit 123. The determination circuit 123 may include an estimation circuit 1231 and a BMI information generation circuit 1232. The BMI information generation circuit 1232 can store a BMI calculation formula. The estimation circuit 1231 can obtain the weight interval information and the height interval information according to the identification result s3. The BMI information generation circuit 1232 generates BMI interval information (i.e., the first body mass index interval information x) by using the BMI calculation formula according to the weight interval information and the height interval information.

Finally, it should be noted that although the output of the identification model of the foregoing embodiment indicates the body mass index interval information, those with ordinary knowledge in the field to which the invention pertains can make the output be an exact body mass index value through appropriate modifications. Correspondingly, the label information used by the identification model in the training phase should be the exact body mass index value. In addition, although the output of the identification model indicates the weight interval information in the first modification and indicates the weight interval information and the height interval information in the second modification, the invention is not limited thereto. Those with ordinary knowledge in the field to which the invention pertains can make the output of the identification model indicate exact weight information, or exact weight information and exact height information through appropriate modifications. Correspondingly, the label information used by the identification model in the training phase should be the exact weight information, or the exact weight information and the exact height information.

In summary, the invention can be used to collect the acceleration information and the angular velocity information and estimate the current body mass index interval information of the user through the identification model. In this way, the current body mass index interval information can be obtained in real time simply by walking without being restricted by the measurement time and site.

1. A body mass index interval estimation device, comprising:
an inertial sensor, suitable for being worn on a body part to detect and obtain first gait information of a user in a traveling state, wherein the first gait information comprises first three-axis acceleration information and first three-axis angular velocity information; and
an arithmetic circuit, configured to cause processed first gait information after a pre-processing to pass an identification model to generate first body mass index interval information,
wherein the identification model is created by performing a training on a sample database, the sample database comprises a plurality of second gait information and a plurality of label information corresponding thereto in a one-to-one manner, and each second gait information in the plurality of second gait information further comprises second three-axis acceleration information and second three-axis angular velocity information.

2. The body mass index interval estimation device of claim 1, wherein the inertial sensor comprises:
an accelerometer, configured to detect and obtain the first three-axis acceleration information of the user in the traveling state; and
an angular velocity meter, configured to detect and obtain the first three-axis angular velocity information of the user in the traveling state.

3. The body mass index interval estimation device of claim 1, wherein each of the second gait information is data information measured at the body part.

4. The body mass index interval estimation device of claim 1, wherein each label information in the plurality of the label information comprises second body mass index interval information.

5. The body mass index interval estimation device of claim 1, wherein the arithmetic circuit is further configured to:
generate first weight interval information according to the first gait information and first height information preset for the user through the identification model; and
obtain the first body mass index interval information according to the first height information and the first weight interval information,
wherein each label information in the plurality of label information comprises second height interval information and second weight interval information.

6. The body mass index interval estimation device of claim 1, wherein the arithmetic circuit is further configured to:
generate first height interval information and first weight interval information according to the first gait information through the identification model; and
obtain the first body mass index interval information according to the first height interval information and the first weight interval information,
wherein each label information in the plurality of label information comprises second height information and second weight interval information.

7. The body mass index interval estimation device of claim 1, wherein the identification model is created by executing, for each of the second gait information in the sample database, steps of:
performing a data extraction on each of the second gait information by using a plurality of finite impulse response filters having windows with different time widths, so as to obtain a plurality of extracted data sets, wherein an amount of data obtained is proportional to the time width of each of the windows;
separately performing a downsampling on each extracted data set in the plurality of extracted data sets, so as to obtain a plurality of downsampled data sets of the same size;
combining the plurality of downsampled data sets to generate a feature matrix;
performing an operation on the feature matrix according to a machine learning algorithm to generate output information, and adjusting a plurality of parameters of the machine learning algorithm according to the output information; and
repeating the steps above until all data in the sample database are used up, and accordingly creating the identification model based on the parameters being adjusted multiple times,
wherein the label information comprises second body mass index interval information, or comprises weight interval information, or comprises height interval information and weight interval information.

8. The body mass index interval estimation device of claim 7, wherein the machine learning algorithm is one of a decision tree, a support vector machine, a multivariable linear regression, a random decision forests, a convolutional neural network and a recurrent neural network.

9. The body mass index interval estimation device of claim 1, further comprising:
a signal pre-processing circuit, configured to perform the pre-processing on each of the first gait information to obtain the corresponding processed first gait information, wherein the pre-processing comprises:
performing a data extraction on each of the first gait information by using a plurality of finite impulse response filters having windows with different time widths, so as to obtain a plurality of extracted data sets, wherein an amount of data obtained is proportional to the time width of each of the windows;
separately performing a downsampling on each extracted data set in the plurality of extracted data sets, so as to obtain a plurality of downsampled data sets of the same size; and
combining the plurality of downsampled data sets to generate a feature matrix, and thereby obtaining the corresponding processed first gait information.

10. The body mass index interval estimation device of claim 1, further comprising: a prompt device, configured to prompt the user with the first body mass index information, wherein the prompt device comprises at least one of a display and a speaker.

11. An operation method of a body mass index interval estimation device, wherein the body mass index interval estimation device comprises an inertial sensor and an arithmetic circuit, and the operation method comprises:
detecting and obtaining first gait information of a user in a traveling state by the inertial sensor worn on a body part, wherein the first gait information comprises first three-axis acceleration information and first three-axis angular velocity information; and
causing processed first gait information after a pre-processing to pass an identification model by the arithmetic circuit to generate first body mass index interval information,
wherein the identification model is created by performing a training on a sample database, the sample database comprises a plurality of second gait information and a plurality of label information corresponding thereto in a one-to-one manner, and each second gait information in the plurality of second gait information further comprises second three-axis acceleration information and second three-axis angular velocity information.

12. The operation method of the body mass index interval estimation device of claim 11, wherein the inertial sensor comprises an accelerometer and an angular velocity meter, wherein the accelerometer is configured to detect and obtain the first three-axis acceleration information of the user in the traveling state, and the angular velocity meter is configured to detect and obtain the first three-axis angular velocity information of the user in the traveling state.

13. The operation method of the body mass index interval estimation device of claim 11, wherein each of the second gait information is data information measured at the body part.

14. The operation method of the body mass index interval estimation device of claim 11, wherein each label information in the plurality of the label information comprises second body mass index interval information.

15. The operation method of the body mass index interval estimation device of claim 11, further comprising:
generating first weight interval information according to the first gait information and first height information preset for the user through the identification model by the arithmetic circuit; and
obtaining the first body mass index interval information according to the first height information and the first weight interval information by the arithmetic circuit,
wherein each label information in the plurality of label information comprises second height interval information and second weight interval information.

16. The operation method of the body mass index interval estimation device of claim 11, further comprising:
generating first height interval information and first weight interval information according to the first gait information through the identification model by the arithmetic circuit; and
obtaining the first body mass index interval information according to the first height interval information and the first weight interval information by the arithmetic circuit,
wherein each label information in the plurality of label information comprises second height information and second weight interval information.

17. The operation method of the body mass index interval estimation device of claim 11, wherein the identification model is created by executing, for each of the second gait information in the sample database, steps of:
performing a data extraction on each of the first gait information by using a plurality of finite impulse response filters having windows with different time widths, so as to obtain a plurality of extracted data sets, wherein an amount of data obtained is proportional to the time width of each of the windows;
separately performing a downsampling on each extracted data set in the plurality of extracted data sets, so as to obtain a plurality of downsampled data sets of the same size;
combining the plurality of downsampled data sets to generate a feature matrix;
performing an operation on the feature matrix according to a machine learning algorithm to generate output information, and adjusting a plurality of parameters of the machine learning algorithm according to the output information,
wherein the steps above are repeated until all data in the sample database are used up, and accordingly creating the identification model based on the parameters being adjusted multiple times,
wherein the label information comprises second body mass index interval information, or comprises weight interval information, or comprises height interval information and weight interval information.

18. The operation method of the body mass index interval estimation device of claim 17, wherein the machine learning algorithm is one of a decision tree, a support vector machine, a multivariable linear regression, a random decision forests, a convolutional neural network and a recurrent neural network.

19. The operation method of the body mass index interval estimation device of claim 11, wherein the pre-processing comprises:
performing a data extraction on each of the first gait information by using a plurality of finite impulse response filters having windows with different time widths, so as to obtain a plurality of extracted data sets, wherein an amount of data obtained is proportional to the time width of each of the windows;
separately performing a downsampling on each extracted data set in the plurality of extracted data sets, so as to obtain a plurality of downsampled data sets of the same size; and
combining the plurality of downsampled data sets to generate a feature matrix, and thereby obtaining the corresponding processed first gait information.

20. The body mass index interval estimation method of claim 11, further comprising: prompting the user with the first body mass index information through at least one of a display and a speaker.
